(19) United States
(12) Patent Application Publication
(10) Pub. No.: US 2011/0224796 A1
(43) Pub. Date: Sep. 15, 2011

(54) IMPLANT FOR FUSING SPINAL COLUMN SEGMENTS

(75) Inventors: Peter Weiland, Nonnweiler (DE); Rudolf Wenzel, Zusch (DE)

(73) Assignee: ADVANCED MEDICAL TECHNOLOGIES AG, Nonnweiler-Braunshausen (DE)

(21) Appl. No.: 13/127,185

(22) PCT Filed: Nov. 5, 2009
(86) PCT No.: PCT/EP2009/064715

§ 371 Date: May 31, 2011

(30) Foreign Application Priority Data
Nov. 7, 2008 (DE) 102008056419.2
Mar. 20, 2009 (DE) 102009014184.7

(51) Int. Cl. A61F 2/44; B05D 5/00; B05D 3/06; B05D 3/02; B05D 3/00

(52) U.S. Cl. 623/17.16; 427/2.26; 427/2.27

(57) ABSTRACT

The invention relates to an optical lens shaped into the form of a shroud and having a light-permeable front side (11) and a side wall (12) adjacent thereto, wherein the side wall (12) and the front side (11) constitute different components of the optical lens (1) that are bound together through injection molding.

The invention relates to a monolithic implant for the fusion of vertebral column segments according to the combination of features of patent claim 1.

Implants for the fusion of vertebral columns are general prior art.

For instance, WO 2006/079356 A1 discloses an implant for the transforaminal interbody fusion of lumbar vertebral column segments. An engagement part is provided on or in the implant which, according to the invention, is constructed as a pivot joint so as to allow an easier implantation process by means of an auxiliary device. Preferably, the implant body is made of a bioelastic plastic material, especially polyetheretherketone (PEEK). The sickle-shaped implant body comprises at least one filling hole between the sickle walls in order to receive a large volume of bone substance.

It also known, however, to produce such implants of metal, especially titanium. Basically, this material allows the surrounding bone and tissue structures to grow together with the implant, but not yet to an extent that would make operating surgeons regard the properties of this implant material as fully developed.

The production of a dental implant made of titanium is described, for instance, in DE 103 15 563 A1. The implant structure includes a prefabricated base body for joining the implant structure to the dental implant and an individually adapted main body. The invention is aimed at forming the main body by sintering or melting a material provided in a powdery form onto the base body in layers by means of laser sintering and/or laser melting. Preferably, the material used is powdery titanium or a titanium containing powder or a powder of a titanium alloy.

Based on the foregoing it is the object of the present invention to provide an implant for the fusion of vertebral column segments, which grows together with the bone and tissue material, which is in direct contact with the surface of the implant, in an improved manner. At the same time, the implant is constructed in such a way that a fast and cost-efficient production of the implant is possible.

The solution to the object is achieved with a monolithic implant for the fusion of vertebral column segments according to the combination of features defined in patent claim 1 and with a method for producing the monolithic implant according to patent claim 23. The dependent claims define at least useful embodiments and further developments.

According to the invention at least parts of the surface of the implant have a structure-forming porosity, and the volume of the implant has a high density. Further, the implant volume includes a number of direction-oriented passages and/or randomly arranged passages pointing in different directions. The passages are surrounded, limited and/or interrupted by stabilizing surfaces that increase the stability of the implant.

The partially structure-forming porosity of the surface of the implant allows surrounding bone, cartilage or tissue material to grow together with the implant more easily. In this context, porosity is not only the simple presence of small channels in the millimeter or micrometer range on a basically smooth surface, but it likewise implies an irregular arrangement of material involving the presence of roughness.

The porosity is only provided on the surface of the implant so that the basic structure of the implant has, at the same time, a high density. Usefully, the inner surfaces of the passages have the structure-forming porosity as well.

Usefully, the partial areas of the implant volume, which include direction-oriented passages and/or randomly arranged passages pointing in different directions, are formed over an as large as possible, stability-uncritical area. Thus, a relatively large implant surface area is obtained, which is provided with holes. Through these holes the bone and tissue material can additionally be joined to the implant.

Usefully, the passages are formed on both sides, that is, passages are provided both on the upper side and lower side of the implant. The upper and lower side of the implant are those surfaces of the implant that point to the adjacent vertebral bodies in the implanted state.

A number of direction-oriented passages which are arranged side by side and/or of randomly arranged passages pointing in different directions are surrounded, limited and/or interrupted by stabilizing surfaces so as to guarantee the stability of the implant despite the presence of the passages. For instance, the edge of the implant may be entirely formed as a stabilizing surface. In other embodiments a stabilizing surface is provided, which divides the total surface area of the number of direction-oriented passages arranged side by side or randomly arranged passages into two partial areas.

Preferably, the described direction-oriented or randomly arranged passages are formed as a honeycomb structure. These hexagonal cavities represent an optimum ratio of the surface of the so produced passage to the stability of the structure that limits the cavity.

It is also possible, however, to form the passages by web connections interleaved into each other or realize the passages in the form of cylindrical channels, wherein the shape of a circular cylinder represents in this regard an embodiment that is easiest to realize in terms of geometry.

Starting from the largest surface side of the monolithic implant the passages usefully extend in a vertical direction. In a particularly preferred embodiment of the present invention the direction-oriented course of the passages is interrupted by at least one clearance. This material-saving construction improves the elasticity of the implant if forces act vertically on the largest surface side.

The clearance may be provided in the area of the center of the implant thickness. In this context, the implant thickness is regarded as the dimension that defines the distance of two vertebrae adjacent to the implant from above and below.

Furthermore, it is possible that the direction-oriented course of the passages is interrupted by at least one stabilizing surface.

However, as was described before, also a random arrangement of the passages is possible. This means that the entirety of the passages do not point in a predetermined direction, but are seemingly arranged completely at random next to each other and one above the other. Such a configuration of the passages comes closest to the natural structure of the cancellous bone. The course of the randomly arranged passages can likewise be interrupted by a clearance or a stabilizing surface.

The lateral faces and/or edges of the implant, which are the first ones to come into contact with the surrounding bone, tissue or cartilage material during the implantation process, preferably have a smoother surface as compared with the surfaces of the implant having a structure-forming porosity. By this, the implantation process is considerably improved because the implant does not “rub” against surrounding bones and tissue pieces and, on the one hand, does not cause damage to the latter and, on the other hand, facilitates the introduction process into the space provided by removing a spinal disc.

Depending on the field of application and operation method used the implant can have different basic shapes.

For instance, kidney shapes, sickle shapes, pin shapes and cuboid shapes are conceivable as basic shapes. The kidney shape as basic shape is used for the fusion of vertebral bodies in the region of the lumbar vertebrae, while the pin shape is suited for the cervical vertebrae or lumbar vertebrae. The sickle shape as basic shape is particularly suited for a so-called TLIF operation technique.

Moreover, the monolithic implant preferably has a slightly wedge-shaped profile, on the one hand, in order to facilitate the implantation process and, on the other hand, in order to comply with the curved shape of the vertebral column.

The surfaces of the implant having a structure-forming porosity have a roughness of 150 μm to 400 μm. A medium roughness of 200 μm was determined to be a particularly preferred degree of roughness.

The monolithic implant further comprises at least one bore for fixing surgical instruments, so that the implant can be inserted easily into the vertebral column.

Moreover, at least one hole is provided in the implant which serves to administer bone replacement material or pastes. The holes are arranged to allow access to the holes by cannulas, syringes or similar auxiliary means in the implanted state. Of particular importance is here the addition of bone replacement material, by means of which it is achieved that the implant and the surrounding vertebral column segments grow together in an enhanced manner.

Depending on the size and chosen basic form the described monolithic implant for the fusion of vertebral column segments is suited for the implantation by means of the posterior lumbar intervertebral fusion operation technique (PLIF) as well as for the implantation by means of the anterior lumbar intervertebral fusion operation technique (ALIF) as well as for the implantation by means of the thoracolumbar intervertebral fusion operation technique (TLIF). Thus, the great advantages of the present invention, namely an enhanced growing together of the implant and the surrounding bone and tissue structures along with an improved stability of the implant during operations can be made use of in the entire spinal area.

In a particularly preferred embodiment the monolithic implant is constructed such that a base body specified with respect to the geometric dimensions of the implant is provided first, so that the stability of the implant and the adaptation to the general anatomical conditions of the partial area of the vertebral column to be attended to are given anytime. In addition, partial areas of the implant are defined as so-called configuration segments, which can be designed variably according to the different customer wishes because these configuration segments are uncritical with respect to stability and can be implanted, for instance, at a smaller size or with a modified geometrical shape. For instance, the operating surgeon can determine the dimensions of the tip of an implant having a pin shape as basic shape. Consequently, the implant can be produced according to the operation habits of the operating surgeon and possible anatomical abnormalities of the patient.

The monolithic implant according to the invention is produced in the course of a sintering method and/or an electron beam melting method. The sintering method and the electron beam melting method each comprise several steps. Initially, the geometrical data of the implant have to be available in a three-dimensional form and processed as cross-sectional data, so that a step-wise fusion of sintering material applied to a base plate in the form of successive horizontal cross-sections is accomplished by means of energy supplied by a beam source and a corresponding cooling after the energy supply and the fusion of a powder layer. Initially, a thin powder layer is applied to the base plate for each individual cross-sectional layer. The sintering powder is dispensed by a powder dispenser and is smoothed by a roller or a doctor blade. The powder layer is then fused in correspondence with the respective dimensions of the cross-sectional layer by means of energy supplied by a beam source, and is cooled afterwards. The energy supplied by the beam source only acts on the powder particles to be solidified, i.e. which represent a material particle of the later implant. Subsequently, the next cross-sectional layer is applied to the lowered base plate and the already fused material and is fused, again, by means of a supply of energy. The processing takes place layer by layer in a vertical direction.

The sintering powder used in the described method is, for instance, a titanium powder. This material is a standard material in the production of implants and is above all characterized by its biocompatibility and the high stability.

It is also possible, however, to use powdery titanium alloys, ceramic powder or polyetheretherketone powder.

The beam source used in the production method is preferably a laser source. The use of an electron beam source is possible as well. If a laser source is used, inter alia, more precise structures can be produced as compared with an electron beam source. The choice with respect to the used beam source thus depends, for instance, on the respective geometrical shape of the monolithic implant.

The aforementioned lateral faces and/or edges of the implant with a smooth surface can be produced after the sintering process in a post-processing step by means of milling machines, polishing machines or turning lathes.

The described production method is particularly suited for the production of several implants having different dimensions in one sintering process. Other than in conventional production methods, e.g. milling, the process according to the sintering method does not require retooling in correspondence with the dimensions of the workpiece to be produced or the loading of different programs for CNC milling. Therefore, it is possible to produce only those implants that are actually needed, and there is no need for producing a plurality of implants with identical dimensions in one operating cycle and storing them subsequently.

If a doctor wants to order a monolithic implant and has special wishes concerning the variably designable configuration segments it is provided by another aspect of the invention that he inputs these dimensions into a predefined mask on a website, and that these data are transmitted to the manufacturer by means of data transmission, where the data are converted to the required cross-sectional data, which are, again by data transmission, transmitted to the sintering plant, where the implant is produced by a sintering method.

After a few days already the orderer receives the produced, customized implant and need not put up with long delivery periods, as is common practice if implants are to be produced according to the customer's wish.

The invention shall be explained in more detail below by means of several embodiment examples and with the aid of figures, wherein:

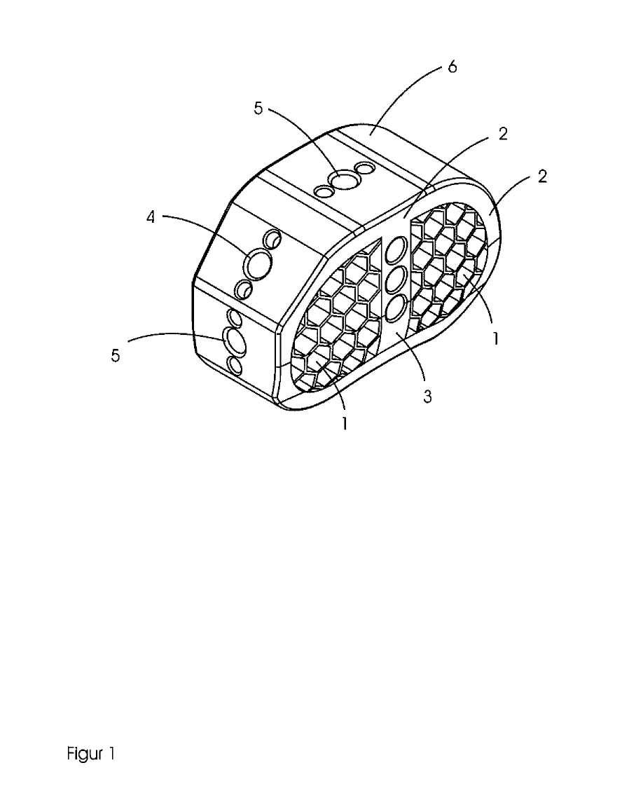

FIG. 1 shows a representation of a monolithic kidney-shaped implant;

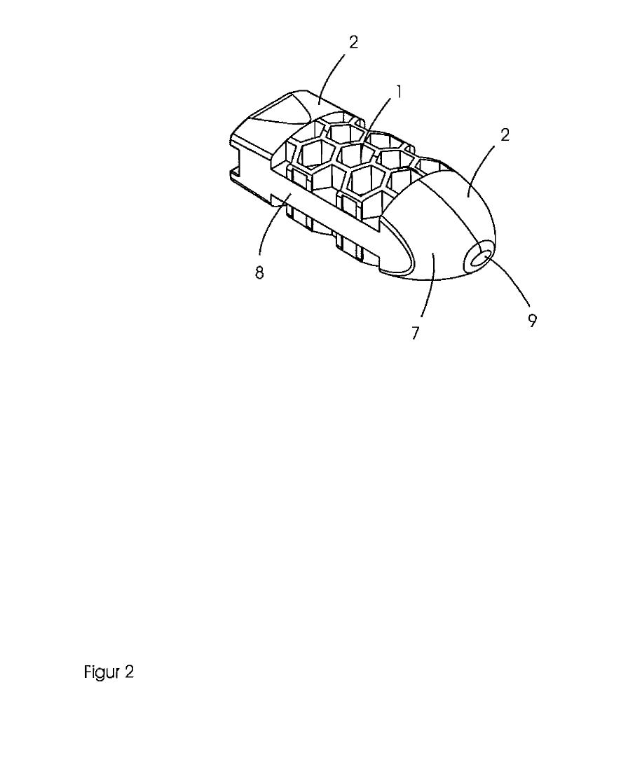

FIG. 2 shows a representation of a monolithic pin-shaped implant;

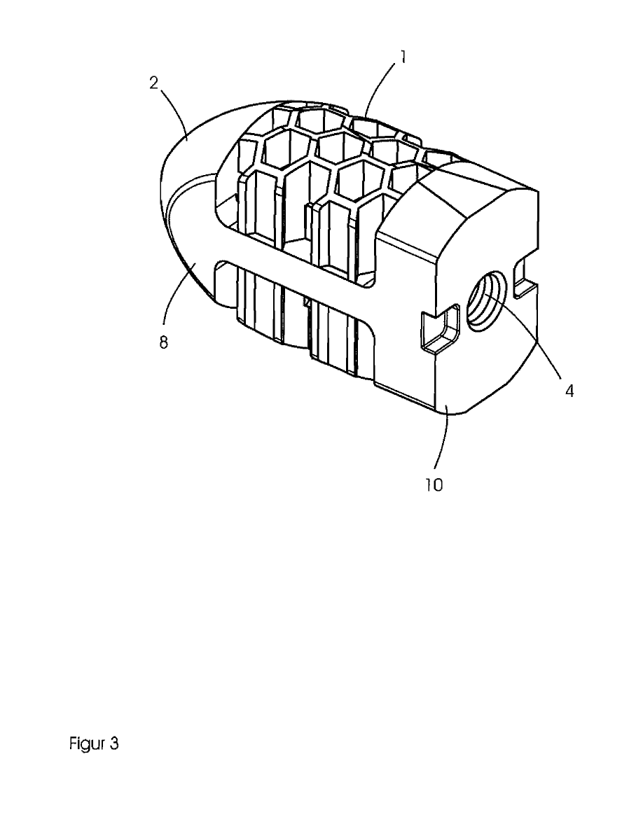

FIG. 3 shows a representation of a monolithic cuboid-shaped implant;

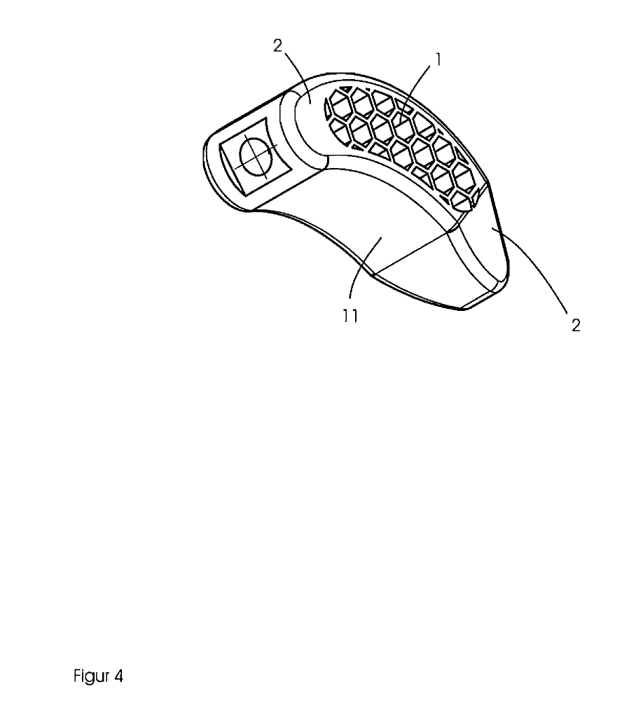

FIG. 4 shows a representation of a monolithic sickle-shaped implant;

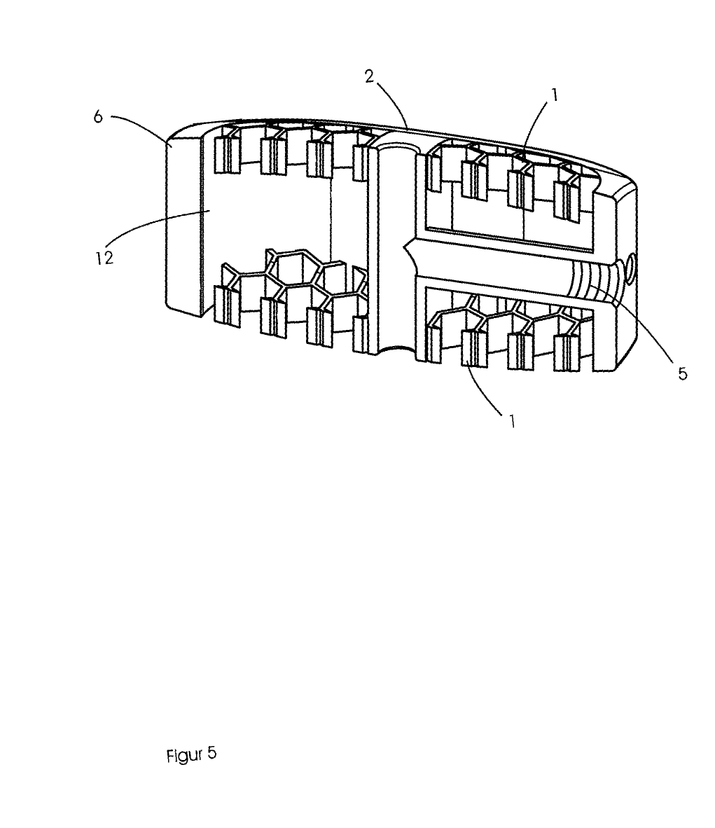

FIG. 5 shows a vertical sectional view of a kidney-shaped monolithic implant; and

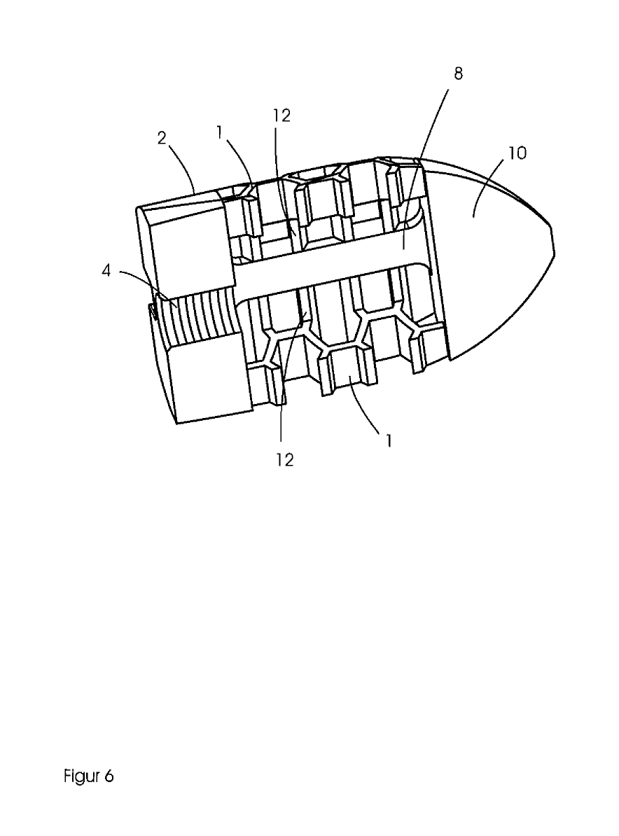

FIG. 6 shows a vertical sectional view of a cuboid-shaped monolithic implant.

FIG. 1 shows a substantially kidney.-shaped monolithic implant for the fusion of vertebral column segments. The direction-oriented passages 1 are well recognizable, which shall be illustrated in the form of a honeycomb structure in the representations to follow.

The direction-oriented passages 1 are surrounded by a stabilizing surface 2, and the total number of the direction-oriented passages 1 are additionally interrupted by another stabilizing surface 2. The structure-forming porosity of the surface is not illustrated in the figures, which is also provided on the inner surfaces of the honeycomb structure.

The stabilizing surfaces 2 have the purpose of providing the implant with sufficient stability, despite the great number of direction-oriented passages 1, for the implant to remain permanently in the human body.

In the illustrated example, the direction-oriented passages 1 extend in a vertical direction, starting from the largest surface side 3 of the monolithic implant.

The bore 4 and holes 5 on the lateral face of the implant are intended, on the one hand, for fixing surgical auxiliary means during the operation and, on the other hand, for administering bone replacement material or pastes. The illustrated kidney shape 6 as basic shape is above all suited if the so-called ALIF operation method is used.

FIG. 2 illustrates a monolithic implant with a pin shape 7 as basic shape. In this embodiment, too, a large portion of the implant volume is provided with direction-oriented passages 1. Noticeable are here the lateral faces 2, which do not completely limit the number of the direction-oriented passages 1 at the lateral area of the implant, but provide for more stability by a narrow web 8 only in the area of the center of the implant thickness. To facilitate the introduction during the implantation process this monolithic implant has a tip 9. In this case, the tip 9 has a smoother surface as compared with the surfaces having the structure-forming porosity, as the tip is the first one to contact the surrounding bone, cartilage and tissue materials during the implantation process. Due to the smooth surface the implantation process can be facilitated additionally. This implant example can be used, above all, for the PLIF operation method.

FIG. 3 shows an embodiment with a cuboid shape 10 as basic shape. As is already illustrated in FIG. 2, the direction-oriented passages 1 have stabilizing surfaces in the form of a web 8 only in the area of the center of the implant thickness.

A sickle shape 11 as basic shape for the monolithic implant is shown in FIG. 4, which can be implanted according to the TLIF operation method:

The sectional view (FIG. 5) of an implant having a kidney shape 6 as basic shape shows that the direction-oriented course of the passages 1 are interrupted by a clearance 12. The clearance 12 serves, on the one hand, the saving of material and, on the other hand, the increased elasticity when the surfaces of the implant are acted on by a force.

As is shown in FIG. 6, the direction-oriented course of the passages 1 may not only be interrupted by a clearance 12, but also by a stabilizing web 8.

In the illustrated/described embodiments, the illustrated/described monolithic implants for the fusion of vertebral column segments were produced by an electron beam melting method or a laser sintering method, with titanium powder being used as sintering powder. As a result of the sintering method surfaces with a structure-forming porosity were obtained. This surface formation also pertains to the inner surfaces of the passages. A roughness of the surface of 42 μm was obtained.

### LIST OF REFERENCE NUMBERS

1 direction-oriented passages
2 stabilizing surface
3 largest surface side
4 bore
5 hole
6 kidney shape as basic shape
7 pin shape as basic shape
8 web
9 tip
10 cuboid shape as basic shape
11 sickle shape as basic shape
12 clearance

1. Monolithic implant for the fusion of vertebral column segments, wherein
at least parts of the surface of the implant have a structure-forming porosity,
the volume of the implant has a high density, further
the implant volume includes a number of direction-oriented passages and/or randomly arranged passages pointing in different directions (1), and
the passages (1) are surrounded, limited and/or interrupted by stabilizing surfaces (2) that increase the stability of the implant.

2. Monolithic implant according to claim 1, wherein
the passages (1) are formed as a honeycomb structure.

3. Monolithic implant according to claim 1, characterized in that
the passages (1) are formed by web connections interleaved into each other.

4. Monolithic implant according to one of claim 1, wherein
the passages (1) are formed by cylindrical channels.

5. Monolithic implant according to claim 1, wherein
starting from the largest surface side (3) of the monolithic implant the passages (1) extend in a vertical direction.

6. Monolithic implant according to claim 1, wherein
the course of the passages (1) is interrupted by at least one clearance (12).

7. Monolithic implant according to claim 6, wherein
the clearance (12) is provided in the area of the center of the implant thickness.

8. Monolithic implant according to claim 1, wherein
the course of the passages (1) is interrupted by at least one stabilizing surface (2).

9. Monolithic implant according to claim 1, wherein
the lateral faces and/or edges of the implant, which are the first ones to come into contact with the surrounding bone, tissue or cartilage material during the implantation process, have a smoother surface as compared with the surfaces of the implant having a structure-forming porosity.

10. Monolithic implant according to claim 1, wherein
the implant substantially has a kidney shape (6) as basic shape.

11. Monolithic implant according to claim 1, wherein
the implant substantially has a pin shape (7) as basic shape.

12. Monolithic implant according to claim 1, wherein
the implant substantially has a cuboid shape (8) as basic shape.

13. Monolithic implant according to claim 1, wherein
the implant substantially has a sickle shape (11) as basic shape.

14. Monolithic implant according to claim 1, wherein
the implant has a wedge-shaped profile.

15. Monolithic implant according to claim 1, wherein
the surfaces of the implant having a structure-forming porosity have a roughness of 150 μm to 400 μm.

16. Monolithic implant according to claim 1, wherein
the surfaces of the implant having a structure-forming porosity have a roughness of 200 μm.

17. Monolithic implant according to claim 1, wherein
the implant comprises at least one bore (4) for fixing surgical instruments.

18. Monolithic implant according to claim 1, wherein
the implant comprises at least one hole (5) for administering bone replacement material or pastes.

19. Monolithic implant according to claim 1, wherein
the implant is used for an implantation carried out by means of the posterior lumbar intervertebral fusion operation technique.

20. Monolithic implant according to claim 1, wherein
the implant is used for an implantation carried out by means of the anterior lumbar intervertebral fusion operation technique.

21. Monolithic implant according to claim 1, wherein
the implant is used for an implantation carried out by means of the thoracolumbar intervertebral fusion operation technique.

22. Monolithic implant according to claim 1, wherein
the implant is comprised of a base body specified with respect to the geometrical dimensions of the implant and configuration segments variably designable according to customer wishes.

23. Method for producing a monolithic implant according to claim 1, wherein
the implant is produced in the course of a sintering method, wherein the three-dimensional form of the monolithic implant is obtained by a step-wise fusion of sintering material applied to a base plate in the form of successive horizontal cross-sections by means of energy supplied by a beam source and a corresponding cooling after the energy supply and the fusion of a powder layer.

24. Method according to claim 23, wherein
the sintering material is a titanium powder.

25. Method according to claim 23, wherein
the sintering material is a powdery titanium alloy.

26. Method according to claim 23, wherein
the sintering material is a ceramic powder or polyetheretherketone powder.

27. Method according to claim 23, wherein
the beam source is a laser.

28. Method according to claim 23, wherein
the beam source is an electron beam source.

29. Method according to claim 23, wherein
the lateral faces and/or edges of the implant with a smooth surface are obtained after the sintering process by a post-processing milling, polishing or turning process.

30. Method according to claim 23, wherein
several implants having different dimensions are produced in one sintering charge.

31. Method according to claim 23, wherein
the three-dimensional dimensions of the implant to be produced, having the dimensions of the configuration segments being variably designable according to customer wishes, are inputted into a mask on a website, are transmitted to a host computer by means of data transmission and are converted to individual cross-sectional data, and, by data transmission, are transmitted to the sintering plant, where the implant is produced by a sintering method.
